(19) United States
(12) Patent Application Publication
(10) Pub. No.: US 2014/0271770 A1
(43) Pub. Date: Sep. 18, 2014

(54) Methods And Compositions For Treating Post-Operative Pain Comprising Clonidine

(71) Applicant: WARSAW ORTHOPEDIC, INC., Warsaw, IN (US)

(72) Inventors: Danielle L. Clay, Collierville, TN (US); William F. McKay, Memphis, TN (US)

(73) Assignee: Warsaw Orthopedic, Inc., Warsaw, IN (US)

(21) Appl. No.: 13/841,961

(22) Filed: Mar. 15, 2013

(51) Int. Cl. A61K 31/4168

(52) U.S. Cl. CPC A61K 31/4168
USPC 424/423; 514/401

(57) ABSTRACT

Effective implantable medical devices for reducing and treating post-operative pain are provided. The implantable medical device comprises clonidine in an amount from about 1 wt. % to about 30 wt. % of the implantable medical device. At least one biodegradable polymer and a pore forming agent in an amount from about 1 wt. % to about 30 wt. % of the implantable medical device is also provided. The implantable medical device is configured to release the clonidine over a period of at least 48 hours. Methods of use are also disclosed.

This application claims the benefit of the filing date of and is a continuation-in-part of U.S. application Ser. No. 12/421,144, filed Apr. 9, 2009, entitled “Methods and Compositions for Treating Post-Operative Pain Comprising Clonidine” which claims the benefit of the filing date of U.S. Provisional Application No. 61/046,277 filed Apr. 18, 2008 and entitled “Methods and Compositions for Treating Post-Operative Pain Comprising Clonidine”. These entire disclosures are hereby incorporated by reference into the present disclosure.

### BACKGROUND

Pain relief is of prime importance to anyone treating patients undergoing surgery. Proper pain relief imparts significant physiological and psychological benefits to the patient. Not only does effective pain relief mean a smoother more pleasant post-operative course (e.g., mood, sleep, quality of life, etc.) but also an earlier discharge from medical, surgical and outpatient facilities.

Pain serves a biological function. It often signals the presence of damage or disease within the body and is often accompanied by inflammation (redness, swelling, and/or burning). In the case of post-operative pain, it may be a result of the surgery, or other treatments such as, for example, management of acute pain following burns or non-surgical trauma. The goal for post-operative pain management is to reduce or eliminate pain and discomfort with medication that causes minimum or no side effects.

The site of the surgery has a profound effect upon the degree of post-operative pain a patient may suffer. In general, operations on the thorax and upper abdomen are more painful than operations on the lower abdomen, which in turn are more painful than peripheral operations on the limbs. However, any operation involving a body cavity, large joint surfaces, the spine or deep tissues should be regarded as painful. In particular, operations on the thorax or upper abdomen may produce widespread changes in pulmonary function, an increase in abdominal muscle tone and an associated decrease in diaphragmatic function. The result will be an inability to cough and clear secretions, which may lead to lung collapse and pneumonia. Prolonged pain can reduce physical activity and lead to venous stasis and an increased risk of deep vein thrombosis and consequently pulmonary embolism. In addition, there can be widespread effects on gut and urinary tract motility, which may lead in turn to post-operative ileus, nausea, vomiting and urinary retention. These problems are unpleasant for the patient and may prolong hospital stay. Many patients that experience moderate to severe post-operative pain, post-traumatic pain and burning pains, often require pain control at least in the first 3 days after trauma or surgery.

One known class of pharmaceuticals to treat post-operative pain is opioids. This class of compounds is well-recognized as being among the most effective type of drugs for controlling post-operative pain. Unfortunately, because opioids are administered systemically, the associated side effects raise significant concerns, including disabling the patient, depressing the respiratory system, constipation, and psychoactive effects such as sedation and euphoria, thereby instituting a hurdle to recovery and regained mobility. Further, because of these side-effects, physicians typically limit the administration of opioids to within the first 24 hours post-surgery. Thus, it would be preferable to use non-narcotic drugs that deliver direct, localized pain control at a surgical site.

One pharmaceutical that is known to the medical profession is clonidine, which is widely recognized as an antihypertensive agent that acts as an agonist on the alpha-2-adrenergic receptor and as a neural receptor agonist. In general, clonidine, also referred to as 2,6-dichloro-N-2-imidazolidinyldenebenzenamine (C.sub.9H.sub.9Cl.sub.2N.sub.3) may be represented by the following chemical structure:

[chemical structure]

However, to date it has not been widely appreciated as an effective treatment for pain including post-operative pain and/or inflammation. Thus, there is a need to develop effective formulations of this compound for this application.

### SUMMARY

Compositions and methods are provided comprising clonidine or its pharmaceutically acceptable salts that are administered in order to treat pain and/or inflammation. The compositions and methods may for example be used to treat post-operative pain from surgical procedures.

In some embodiments, there is an implantable medical device for reducing or treating post-operative pain in a patient in need of such treatment, the implantable medical device comprising clonidine in an amount from about 1 wt. % to about 30 wt. % of the implantable medical device. At least one biodegradable polymer and a pore forming agent in an amount from about 1 wt. % to about 30 wt. % of the implantable medical device is provided. The implantable medical device is configured to release the clonidine over a period of at least 48 hours.

In some embodiments, there is an implantable medical device for treating post-operative pain in a patient in need of such treatment, the implantable medical device comprising clonidine hydrochloride in an amount from about 3 wt. % to about 5 wt. % of the implantable medical device. Poly(L-lactide-co-caprolactone) comprising about 85 wt. % to about 95 wt. % of the implantable medical device and mannitol in an amount from about 1 wt. % to about 10 wt. % of the implantable medical device is provided. The implantable medical device is configured to release the clonidine hydrochloride over a period of at least 2 to about 14 days.

In some embodiments, there is a method for treating post-operative pain in a patient in need of such treatment, the method comprising: administering an implantable medical device beneath the skin of a patient, the implantable medical device comprising clonidine in an amount from about 1 wt. % to about 30 wt. % of the implantable medical device, and at least one biodegradable polymer and a pore forming agent in an amount from about 1 wt. % to about 30 wt. % of the implantable medical device, wherein the implantable medical device is configured to release the clonidine over a period of at least 48 hours.

In some embodiments, the device is a drug depot that may: (i) consist of only the clonidine (or one or more of its pharmaceutically acceptable salts) and the biodegradable polymer(s) and the pore forming agent or plasticizer; or (ii) consist essentially of the clonidine (and/or one or more of its pharmaceutically acceptable salts) and the biodegradable polymer(s) and the pore forming agent or plasticizer; or (iii) comprise the clonidine (and/or one or more of its pharmaceutically acceptable salts), and the biodegradable polymer(s) and one or more other active ingredients, surfactants, excipients or other ingredients or combinations thereof. When there are other active ingredients, surfactants, excipients or other ingredients or combinations thereof in the formulation, in some embodiments these other compounds or combinations thereof comprise less than 70 wt. %, less than 60 wt. %, less than 50 wt. %, less than 40 wt. %, less than 30 wt. %, less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. %.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 provides a front view of a surgical site where implantable medical devices are implanted in a pig model to determine the efficacy of the implantable medical devices for treating post-operative pain. The implantable medical devices are drug depots and an implant site may comprise of 1, 2, 3, 4, 5, 6, 7, 8, 9 or 10 drug depots. The drug depots triangulate around the surgical site.

FIG. 2 is a graphical depiction of dose responses for pain relief over a period of 7 days using Von Frey methodology.

FIG. 3 is a graphical depiction of the mean plasma concentration of clonidine over a period of 14 days.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

### DEFINITIONS

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

A “drug depot” is the composition in which the clonidine is administered to the body. Thus, a drug depot may comprise a physical structure (e.g., device) to facilitate implantation and retention in a desired site (e.g., a disc space, a spinal canal, a tissue of the patient, particularly at or near a site of post-operative pain, etc.). The drug depot (e.g., device) may also comprise the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include one or more than one therapeutic agent, wherein exemplary combinations of therapeutic agents can include a combination of two or more drugs. The drug provides a concentration gradient of the therapeutic agent for delivery to the site. In various embodiments, the drug depot (e.g., device) provides an optimal drug concentration gradient of the therapeutic agent at a distance of up to about 0.01 cm to about 20 cm from the administration site and comprises clonidine. A drug depot (e.g., device) may also include a pump, pellet, strip and/or fiber.

A “therapeutically effective amount” or “effective amount” is such that when administered, the drug results in alteration of the biological activity, such as, for example, inhibition of inflammation, reduction or alleviation of pain or spasticity, improvement in the condition through muscle relaxation, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the formulation is designed for immediate release. In other embodiments the formulation is designed for sustained release. In other embodiments, the formulation comprises one or more immediate release surfaces and one or more sustained release surfaces.

A “depot” includes but is not limited to capsules, microspheres, microparticles, microcapsules, micro-depot particles, nanospheres, nanoparticles, coating, matrices, wafers, pills, pellets, emulsions, liposomes, micelles, gels, strips, fibers or other pharmaceutical delivery compositions or a combination thereof. Suitable materials for the depot (e.g., device) are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

The “depot” of the present application provides a 3-D structure of interconnecting pores, which acts as a pliant scaffold for cell migration and/or drug release.

As used herein, “depot” refers to any flexible structure that can be stretched between two points and includes, without limitation, traditional depot material, single or multiple stranded threads, or a mesh structure. A depot may also be a strap-like structure with a number of holes in it. A “depot” may also take the form of an acellular membrane or other biologic tissue augment, which may provide a scaffold or support matrix for cellular ingrowth to allow soft tissue to reconstruct itself. Depots may include silk, nylon, linen, cotton, chromic gut, plain gut, cat gut, vicryl, polyglactin, polyester, polypropylene, stainless steel, synthetic polymers having lactic acid or glycolic acid ester linkages subject to hydrolytic degradation to non-toxic tissue compatible absorbable components, including polylactic acid or polyglycolic acid. The depot may be absorbable or non-absorbable.

The term “biodegradable” includes that all or parts of the drug depot (e.g., device) will degrade over time by the action of enzymes, by hydrolytic action and/or by other similar mechanisms in the human body. In various embodiments, “biodegradable” includes that the depot (e.g., device) can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot (e.g., device) will be broken down and absorbed within the human body, for example, by a cell or tissue. “Biocompatible” means that the depot (e.g., device) will not cause substantial tissue irritation or necrosis at the target tissue site.

In some embodiments, the drug depot (e.g., device) has pores that allow release of the drug from the depot (e.g., device). The drug depot (e.g., device) will allow fluid in the depot (e.g., device) to displace the drug. However, cell infiltration into the depot (e.g., device) will be prevented by the size of the pores of the depot (e.g., device). In this way, in some embodiments, the depot (e.g., device) should not function as a tissue scaffold and allow tissue growth. Rather, the drug depot (e.g., device) will solely be utilized for drug delivery. In some embodiments, the pores in the drug depot (e.g., device) will be less than 250 to 500 microns. This pore size will prevent cells from infiltrating the drug depot (e.g., device) and laying down scaffolding cells. Thus, in this embodiment, the drug will elute from the drug depot (e.g., device) as fluid enters the drug depot (e.g., device), but cells will be prevented from entering. In some embodiments, where there are little or no pores, the drug will elute out from the drug depot (e.g., device) by the action of enzymes, by hydrolytic action and/or by other similar mechanisms in the human body.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot, or a device or component thereof, or as the result of metabolic transformation or dissolution of the therapeutic agent(s) or conjugates of therapeutic agent(s).

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

The two types of formulations (sustain release and immediate release) may be used in conjunction. The sustained release and immediate release may be in one or more of the same depots. In various embodiments, the sustained release and immediate release may be part of separate depots. For example a bolus or immediate release formulation of clonidine may be placed at or near the target site and a sustain release formulation may also be placed at or near the same site. Thus, even after the bolus becomes completely accessible, the sustain release formulation would continue to provide the active ingredient for the intended tissue.

In various embodiments, the depot can be designed to cause an initial burst dose of therapeutic agent within the first 24 hours to 72 hours after implantation. “Initial burst” or “burst effect” “burst release” or “bolus dose” refers to the release of therapeutic agent from the depot (e.g., device) during the first 24 hours to 72 hours after the depot (e.g., device) comes in contact with an aqueous fluid (e.g., interstitial fluid, synovial fluid, cerebral spinal fluid, etc.). The “burst effect” is believed to be due to the increased release of therapeutic agent from the depot. In some embodiments, the depot has one or more burst release surfaces that releases about 10%, 15%, 20%, 25%, 30%, 35%, 45%, to about 50% of the drug over 24 or 48 hours.

In alternative embodiments, the depot is designed to avoid or reduce this initial burst effect (e.g., by applying an outer polymer coating to the depot).

“Treating” or “treatment” of a disease or condition refers to executing a protocol that may include administering one or more drugs to a patient (human, other normal or otherwise or other mammal), in an effort to alleviate signs or symptoms of the pain or condition. Alleviation can occur prior to signs or symptoms of the pain or condition appearing, as well as after their appearance. Thus, treating or treatment includes preventing or prevention of pain or an undesirable condition. In addition, treating or treatment does not require complete alleviation of signs or symptoms, does not require a cure, and specifically includes protocols that have only a marginal effect on the patient. “Reducing pain and/or inflammation” includes a decrease in pain and/or inflammation and does not require complete alleviation of pain and/or inflammation signs or symptoms, and does not require a cure. In various embodiments, reducing pain and/or inflammation includes even a marginal decrease in pain and/or inflammation. By way of example, the administration of the effective dosage of clonidine may be used to prevent, treat or relieve the symptoms of pain and/or inflammation for different surgical procedures or conditions. These surgical procedures/conditions may comprise post-operative pain and/or acute pain relief.

The term “implantable” as utilized herein refers to a biocompatible depot (e.g., implantable medical device) retaining potential for successful placement within a mammal. The expression “implantable depot”, “implantable medical device” and expressions of the like import as utilized herein refers to an object implantable through surgery, injection, or other suitable means whose primary function is achieved either through its physical presence or mechanical properties.

“Localized” delivery includes delivery where one or more drugs are deposited within a tissue, for example, a nerve root of the nervous system or a region of the brain, or in close proximity (within about 0.1 cm, or preferably within about 10 cm, for example) thereto. For example, the drug dose delivered locally from the depot may be, for example, 10%, 20%, 30%, 40%, 50%, 60%, 70%, 80%, 90%, 95%, 99%, or 99.9% less than the oral dosage or injectable dose. In turn, systemic side effects, such as for example, liver transaminase elevations, hepatitis, liver failure, myopathy, constipation, etc. may be reduced or eliminated.

The term “mammal” refers to organisms from the taxonomy class “mammalian,” including but not limited to humans, other primates such as chimpanzees, apes, orangutans and monkeys, rats, mice, cats, dogs, cows, horses, etc.

The phrase “pain management medication” includes one or more therapeutic agents that are administered to prevent, alleviate or remove pain entirely. These include anti-inflammatory agents, muscle relaxants, analgesics, anesthetics, narcotics, and so forth, and combinations thereof.

The phrase “release rate profile” refers to the percentage of active ingredient that is released over fixed units of time, e.g., mcg/hr, mcg/day, 10% per day for ten days, etc. As persons of ordinary skill know, a release rate profile may, but need not, be linear. By way of a non-limiting example, the depot may be a ribbon-like depot that releases the clonidine over a period of time.

The term “solid” is intended to mean a rigid material, while, “semi-solid” is intended to mean a material that has some degree of flexibility, thereby allowing the depot (e.g., device) to bend and conform to the surrounding tissue requirements.

“Targeted delivery system” provides delivery of one or more drugs depots (e.g., devices) at or near the target site as needed for treatment of pain, inflammation or other disease or condition.

In various embodiments, pain results from “post-surgical pain” or “post-operative pain” or “surgery-induced pain”, which are used herein interchangeably, and refer to pain arising in the recovery period of seconds, minutes, hours, days or weeks following a surgical procedure (e.g., hernia repair, orthopedic or spine surgery, etc.). Surgical procedures include any procedure that penetrates beneath the skin and causes pain and/or inflammation to the patient. Surgical procedure also includes arthroscopic surgery, an excision of a mass, spinal fusion, thoracic, cervical, or lumbar surgery, pelvic surgery or a combination thereof.

The term “pain management medication” includes one or more therapeutic agents that are administered to reduce, prevent, alleviate or remove pain entirely. These include anti-inflammatory agents, muscle relaxants, analgesics, anesthetics, narcotics, etc., or combinations thereof.

In various embodiments, the post-surgical pain or post-operative pain or surgery-induced pain, is accompanied by inflammation. Inflammation can be an acute response to trauma or surgery. When tissues are damaged, TNF-α attaches to cells to cause them to release other cytokines that cause inflammation. The purpose of the inflammatory cascade is to promote healing of the damaged tissue, but once the tissue is healed the inflammatory process does not necessarily end. Left unchecked, this can lead to degradation of surrounding tissues and associated pain. Thus, pain can become a disease state in itself. That is, when this pathway is activated, inflammation and pain ensue. Often a vicious and seemingly endless cycle of insult, inflammation, and pain sets in.

The abbreviation “DLG” refers to poly(DL-lactide-co-glycolide).

The abbreviation “DL” refers to poly(DL-lactide).

The abbreviation “LG” refers to poly(L-lactide-co-glycolide).

The abbreviation “CL” refers to polycaprolactone.

The abbreviation “DLCL” refers to poly(DL-lactide-co-caprolactone).

The abbreviation “LCL” refers to poly(L-lactide-co-caprolactone).

The abbreviation “G” refers to polyglycolide.

The abbreviation “PEG” refers to poly(ethylene glycol).

The abbreviation “PLGA” refers to poly(lactide-co-glycolide) also known as poly(lactic-co-glycolic acid), which are used interchangeably.

The abbreviation “PLA” refers to polylactide.

The abbreviation “PEA” refers to poly(ester)amides.

The abbreviation “POE” refers to poly(orthoester). The above polymers or combination of polymers can be in the drug depot (e.g., device).

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

### Clonidine Compounds

When referring to clonidine, unless otherwise specified or apparent from context it is understood that the inventors are also referring to pharmaceutically acceptable salts. One well-known commercially available salt for clonidine is its hydrochloride salt. Some other examples of potentially pharmaceutically acceptable salts include those salt-forming acids and bases that do not substantially increase the toxicity of a compound, such as, salts of alkali metals such as magnesium, potassium and ammonium, salts of mineral acids such as hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like.

Further, when referring to clonidine, the active ingredient may not only be in the salt form, but also in the base form (e.g., free base). In various embodiments, if it is in the base form, it may be combined with polymers under conditions in which there is not severe polymer degradation, as may be seen upon heat or solvent processing that may occur with PLGA or PLA. By way of a non-limiting example, when formulating clonidine with poly(orthoesters) it may be desirable to use the clonidine base formulation. By contrast, when formulating clonidine with PLGA, it may be desirable to use the HCl salt form. In some embodiments, the clonidine may be incorporated into a polymer core with a polymer and then coated with the same or different polymer.

Pharmaceutically acceptable salts of clonidine include salts prepared from pharmaceutically acceptable non-toxic bases or acids including inorganic or organic bases, inorganic or organic acids and fatty acids. Salts derived from inorganic bases include aluminum, ammonium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethyl amine, tripropylamine, tromethamine, and the like. When the compound of the current application is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Fatty acid salts may also be used, e.g., fatty acid salts having greater than 2 carbons, greater than 8 carbons or greater than 16 carbons, such as butyric, caprioc, caprylic, capric, lauric, mystiric, palmitic, stearic, arachidic or the like.

In some embodiments, in order to reduce the solubility of the clonidine to assist in obtaining a controlled release depot (e.g., device) effect, clonidine is utilized as the free base or utilized in a salt which has relatively lower solubility. For example, the present application can utilize an insoluble salt such as a fatty acid salt. Representative fatty acid salts include salts of oleic acid or linoleic acid. In preferred embodiments fatty acid salts with between 8 to 20 carbons are used to produce salts with low solubility, such as clonidine palmeate and clonidine stearate. Most preferably, fatty acid salts with between 12 to 18 carbons are used. Other embodiments can utilize a lipid soluble salt of clonidine.

In some embodiments, the clonidine compound is released locally from the depot at a dose of from about 40 mcg/day for 7 days or between about 80 ug and 250 ug per day for a period of about 2 to 14 days about. In some embodiments, the initial burst or bolus release is about 2 to 20 times higher from 1 hour to about two weeks than the sustained release daily dose released from the depot.

In some embodiments, the depot comprises clonidine that is in the depot in an amount of from about 0.1% to about 75% by weight. In some embodiments, the clonidine is in the depot in an amount from about 1% to about 30% by weight. In some embodiments, the clonidine is in the depot in an about from about 30% to about 75% by weight. In some embodiments, the clonidine is in the depot in an amount from about 75% to about 99.1% by weight.

In some embodiments, the clonidine can be in powdered form having a particle sizes predominantly in a range from about 3.5 to about 10 micrometers that can be reconstituted with the polymer for delivery. In some embodiments, the clonidine can have a particle size from about 10 to about 20 microns. In various embodiments, the drug particle size (e.g., clonidine compound) is from about 5 to 30 micrometers, however, in various embodiments ranges from about 1 micron to 250 microns may be used.

In some embodiments, the implantable drug depot comprising clonidine hydrochloride in an amount from about 3 wt. % to about 5 wt. % of the implantable medical device, poly(L-lactide-co-caprolactone comprising about 85 wt. % to about 95 wt. % of the implantable device, and mannitol in an amount from about 2.5 wt. % to about 10 wt. % of the implantable drug depot. The implantable drug depot is configured to release the clonidine hydrochloride over a period of at least 2 to about 14 days.

In some embodiments, at least 75% of the particles have a size from about 10 micrometer to about 200 micrometers. In some embodiments, at least 85% of the particles have a size from about 10 micrometer to about 200 micrometers. In some embodiments, at least 95% of the particles have a size from about 10 micrometer to about 200 micrometers. In some embodiments, all of the particles have a size from about 10 micrometer to about 200 micrometers.

In some embodiments, at least 75% of the particles have a size from about 20 micrometer to about 180 micrometers. In some embodiments, at least 85% of the particles have a size from about 20 micrometers to about 180 micrometers. In some embodiments, at least 95% of the particles have a size from about 20 micrometer to about 180 micrometers. In some embodiments, all of the particles have a size from about 20 micrometer to about 180 micrometers.

In some embodiments, there is a pharmaceutical formulation comprising clonidine, wherein the clonidine is in a mixture of clonidine hydrochloride and clonidine base and the mixture comprises from about 0.1 wt. % to about 30 wt. % of the formulation and a biodegradable polymer comprises at least 70% of the formulation. In some embodiments, the polymer in this formulation is poly(L-lactide-co-caprolactone).

### Excipients and Therapeutic Agents

The depot may comprise other therapeutic agents in addition to the clonidine compound as well. These therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents include IL-1 inhibitors, such Kineret® (anakinra) which is a recombinant, non-glycosylated form of the human interleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogs (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as glucocorticoids, antioxidants, such as dithiocarbamate, and other compounds, such as, for example, sulfasalazine.

Examples of therapeutic agents suitable for use also include, but are not limited to an anti-inflammatory agent, an analgesic agent, or an osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine [2-hydroxy-5-[-4-[C2-pyridinylamino)sulfonyl]azo]benzoic acid, sulindac, tepoxalin or tolmetin; as well as antioxidants, such as dithiocarbamate, steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable anabolic growth or anti-catabolic growth factors include, but are not limited to, a bone morphogenetic protein, a growth differentiation factor (e.g., GDF-5), a LIM mineralization protein, CDMP or progenitor cells or a combination thereof.

Suitable analgesic agents include, but are not limited to, acetaminophen, bupivacaine, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papavereturn, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

The therapeutic agent in the depot may include, but is not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs; the TGF-β superfamily, including TGF-β1, 2 or 3; osteoid-inducing factor (OIF); angiogenin(s); endothelins; hepatocyte growth factor or keratinocyte growth factor; members of the bone morphogenetic proteins (BMP's) BMP-1, BMP-3, BMP-2; OP-1, BMP-2A, BMP-2B, or BMP-7; HBGF-1 or HBGF-2; growth differentiation factors (GDF's); members of the hedgehog family of proteins, including indian, sonic and desert hedgehog; ADMP-1; other members of the interleukin (IL) family; or members of the colony-stimulating factor (CSF) family, including CSF-1, G-CSF, and GM-CSF, or isoforms thereof; or VEGF, NELL-1 (neural epidermal growth factor-like 1), CD-RAP (cartilage-derived retinoic acid-sensitive protein) or combinations thereof.

In some embodiments, the depot comprises osteogenic proteins. Exemplary osteogenic proteins include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, CDMP-1, CDMP-2, CDMP-3, DPP, Vg-1, Vgr-1, 60A protein, NODAL, UNIVIN, SCREW, ADMP, NEURAL, and TGF-beta. As used herein, the terms “morphogen,” “bone morphogen,” “BMP,” “osteogenic protein” and “osteogenic factor” embrace the class of proteins typified by human osteogenic protein 1 (hOP-1).

Exemplary growth factors include, but are not limited to, members of the transforming growth factor beta family, including bone morphogenetic protein 2 (BMP-2); bone morphogenetic protein 4 (BMP-4); and transforming growth factors beta-1, beta-2, and beta-3 (potent keratinocyte growth factors). Other useful members of the transforming growth factor beta family include BMP-3, BMP-5, BMP-6, BMP-9, DPP, Vgl, Vgr, 60A protein, GDF-1, GDF-3, GDF-5, GDF-6, GDF-7, CDMP-1, CDMP-2, CDMP-3, BMP-10, BMP-11, BMP-13, BMP-15, Univin, Nodal, Screw, ADMP, Neural, and amino acid sequence variants thereof. Other growth factors include epidermal growth factor (EGF), which induces proliferation of both mesodermal and ectodermal cells, particularly keratinocytes and fibroblasts; platelet-derived growth factor (PDGF), which exerts proliferative effects on mesenchymal cells; fibroblast growth factor (FGF), both acidic and basic; and insulin-like growth factor 1 (IGF-1) or 2 (IGF-2), which mediate the response to growth hormone, particularly in bone growth. Further growth factors include osteogenic proteins. A particularly preferred osteogenic protein is OP-1, also known as bone morphogenetic protein 7 (BMP-7). OP-1 is a member of the transforming growth factor beta gene superfamily.

The clonidine may also be administered with non-active ingredients. These non-active ingredients may have multi-functional purposes including the carrying, stabilizing and controlling the release of the therapeutic agent(s). The sustained release process, for example, may be by a solution-diffusion mechanism or it may be governed by an erosion-sustained process. Typically, the depot (e.g., device) will be a solid or semi-solid formulation comprised of a biocompatible material that can be biodegradable.

Excipients, plasticizers, and/or pore forming agents may be formulated with clonidine in addition to the biodegradable polymer. Plasticizers and/or pore forming agents impart malleability to the resulting formulations. Additionally, plasticizers and/or pore forming agents make the biodegradable polymer more porous after implantation, creating a controlled burst release of the clonidine. In some embodiments, the plasticizers and/or pore forming agents include but are not limited to MgO (e.g., 1 wt. %), mPEG, propylene glycol, mannitol, trehalose, TBO-Ac, Span-65, Span-85, pluronic F127, sorbitol, xylitol, isomalt, erithritol, cyclodextrin, maltodextrin, pluronic F68, CaCl, dextran, dextran sulphate, dextran phosphate, hydroxypropylcellulose, ethylcellulose, PEG 1500, PEG 400, PEG3350, acetyl tributyl citrate, butyl benzyl phthalate, butyl phthalyl butyl glycolate, dibutyl phthalate, dibutyl sebacate, diethyl phthalate, diethylene glycol dibenzoate, dipropylene glycol, dipropylene glycol dibenzoate, ethyl phthalyl ethyl glycolate, ethyl-p-toluene sulfonamide, hexylene glycol, methyl phthalyl ethyl glycolate, polyoxyethylene aryl ether and tributoxyethyl phthalate or combinations thereof. Other plasticizers that may be utilized include esters of ortho, iso and terephthalate, esters of citric acid, esters of 1,2-, 1,3- and 1,4-cyclohexane dicarboxylic acid, and anhydrides or combinations thereof. Diluent plasticizers such as 2-ethylhexyl benzoate, isodecyl benzoate or 2,2,4-trimethyl-1,3-pentanediol diisobutyrate may also be used to control rheology and other properties. Diluents which may be utilized include aromatic hydrocarbon, cycloaliphatic hydrocarbon, and aliphatic hydrocarbons. In some embodiments, the particle size of the plasticizer and/or excipient (e.g., mannitol) is about 1 to about 250 microns, about 1 to about 100 microns or about 1 to about 10 microns. In various embodiments, Dv 10 or Dv 50 is about 1 to about 100 microns. In some embodiments, the plasticizer and or pore forming agent is amorphous and/or crystalline. In various embodiments, mannitol is crystalline and not amorphous.

In some embodiments, the pore forming agent comprises from about 0.5 wt. % to about 50 wt. % of the formulation. In some embodiments, the pore forming agent comprises from about 1.0 wt. % to about 40 wt. % of the formulation. In some embodiments, the pore forming agent comprises from about 1 wt. % to about 30 wt. % of the formulation. In some embodiments, the pore forming agent comprises from about 5 wt. % to about 20 wt. % of the formulation. In some embodiments, the pore forming agent comprises from about 2 wt. % to about 10 wt. % of the formulation. In some embodiments, the pore forming agent comprises from about 2.5 wt. % to about 30 wt. % of the formulation. In some embodiments, the pore forming agent comprises from about 1 wt. % to about 3 wt. % of the formulation.

In particular embodiments, the pore forming agent or plasticizer comprises 1%, 1.5%, 2%, 2.5%, 3%, 3.5%, 4%, 4.5%, 5%, 5.5%, 6%, 6.5%, 7%, 7.5%, 8%, 8.5%, 9%, 9.5% to about 10% % w/w, w/v, or v/v of the composition.

In some embodiments, the other excipients comprise from about 0.001 wt. % to about 50 wt. % of the formulation. In some embodiments, the excipients comprise from about 0.001 wt. % to about 40 wt. % of the formulation. In some embodiments, the excipients comprise from about 1 wt. % to about 30 wt. % of the formulation. In some embodiments, the excipients comprise from about 5 wt. % to about 20 wt. % of the formulation. In some embodiments, the excipients comprise from about 2 wt. % to about 10 wt. % of the formulation. In some embodiments, the excipients comprise from about 2.5 wt. % to about 30 wt. % of the formulation. In some embodiments, the excipients comprise from about 1 wt. % to about 3 wt. % of the formulation.

The clonidine or its pharmaceutically acceptable salt may be administered with a muscle relaxant. Exemplary muscle relaxants include by way of example and not limitation, alcuronium chloride, atracurium bescylate, carbamate, carbolonium, carisoprodol, chlorphenesin, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

### Drug Depot

In some embodiments, the implantable medical device comprises a drug depot. In various embodiments, a plurality of drug depots (e.g., pellets) can be administered to a surgical site. In some embodiments, a plurality of drug depots are provided (e.g., in a kit) and administered to a surgical site and triangulate and/or surround the site to treat post-operative pain. In various embodiments, a plurality of drug depots comprise about 1, 2, 3, 4, 5, 6, 7, 8, 9 or 10 drug depots.

In some embodiments, the drug depot has a melting point or glass transition temperature close to or higher than body temperature, but lower than the decomposition or degradation temperature of the therapeutic agent. However, the pre-determined erosion of the depot (e.g., device) material can also be used to provide for slow release of the loaded therapeutic agent(s). Non-biodegradable polymers include but are not limited to PVC and polyurethane. In some embodiments, a plasticizer is used to lower glass translation temperature in order to affect stability of the device.

In various embodiments, the depot comprises clonidine and a biodegradable polymer in amorphous, crystalline or semicrystalline form; where the crystalline form may include polymorphs, solvates or hydrates.

In some embodiments, the depot has a modulus of elasticity in the range of about 1×10² to about 6×10⁵ dyn/cm², or 2×10⁴ to about 5×10⁵ dyn/cm², or 5×10⁴ to about 5×10⁵ dyn/cm². In some embodiments, the device is in the form of a solid.

In some embodiments, the clonidine compound is administered in a device that is solid or in semi-solid form. The solid or semi-solid form of the device may have a pre-dosed viscosity in the range of about 1 to about 2000 centipoise (cps), 1 to about 200 cps, or 1 to about 100 cps. After the solid or semi-solid device is administered to the target site, the viscosity of the semi-solid or solid depot will increase and the semi-solid will have a modulus of elasticity in the range of about 1×10² to about 6×10⁵ dynes/cm², or 2×10⁴ to about 5×10⁵ dynes/cm², or 5×10⁴ to about 5×10⁵ dynes/cm².

In various embodiments, the semi-solid or solid depot may comprise a polymer having a molecular weight (MW), as shown by the inherent viscosity, from about 0.10 dL/g to about 1.2 dL/g or from about 0.20 dL/g to about 0.50 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.55 to about 0.6 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, or about 1.8 to about 2.1 dL/g.

In some embodiments, the depot may comprise an 8% loaded 60:40 LCL 5A with a 6.5% content having a 0.4 mm diameter; an 8% loaded 60:40 LCL 5A with a 6.6% content having a 0.8 mm diameter; or a 16% loaded 60:40 LCL 5A with a 13.2% content having a 0.6 mm diameter.

In some embodiments, the depot may not be fully biodegradable. For example, the device may comprise polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon device, glass device, plastics, ceramics, methacrylates, poly(N-isopropylacrylamide), PEO-PPO-PEO (pluronics) or combinations thereof. Typically, these types of matrices may need to be removed after a certain amount of time.

In some instances, it may be desirable to avoid having to remove the depot after use. In those instances, the depot may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (e.g., device) (homogeneous or bulk erosion).

In various embodiments, the depot (e.g., device) may comprise a bioerodible, a bioabsorbable, and/or a biodegradable biopolymer that may provide immediate release, or sustained release of the clonidine. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), poly(orthoester)s (POE), poly(esteramide)s, polyaspirins, polyphosphagenes, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof.

In some embodiments, the depot comprises biodegradable polymers comprising wherein the at least one biodegradable polymer comprises one or more of poly(lactide-co-glycolide) (PLGA), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide) or a combination thereof.

In some embodiments, the drug depot comprises at least one biodegradable material in a wt % of about 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 76%, 75%, 74%, 73%, 72%, 71%, 70%, 65%, 60%, 55%, 50%, 45%, 35%, 25%, 20%, 15%, 10%, or 5% based on the total weight of the depot and the remainder is active and/or inactive pharmaceutical ingredients.

In some embodiments, the at least one biodegradable polymer comprises poly(L-lactide-co-caprolactone). In various embodiments, there is at least 95% poly(L-lactide-co-caprolactone); at least 90% poly(L-lactide-co-caprolactone); at least 85% poly(L-lactide-co-caprolactone); at least 80% poly(L-lactide-co-caprolactone); at least 75% poly(L-lactide-co-caprolactone); at least 70% poly(L-lactide-co-caprolactone); at least 65% poly(L-lactide-co-caprolactone); at least 60% poly(L-lactide-co-caprolactone); at least 55% poly(L-lactide-co-caprolactone); at least 50% poly(L-lactide-co-caprolactone); at least 45% poly(L-lactide-co-caprolactone); at least 40% poly(L-lactide-co-caprolactone); at least 35% poly(L-lactide-co-caprolactone); at least 30% poly(L-lactide-co-caprolactone); at least 25% poly(L-lactide-co-caprolactone); at least 20% poly(L-lactide-co-caprolactone); at least 15% poly(L-lactide-co-caprolactone); at least 10% poly(L-lactide-co-caprolactone); or at least 5% poly(L-lactide-co-caprolactone).

In some embodiments, at least 75% of the biodegradable polymer particles have a size from about 10 micrometer to about 200 micrometers. In some embodiments, at least 85% of the particles have a size from about 10 micrometer to about 200 micrometers. In some embodiments, at least 95% of the particles have a size from about 10 micrometer to about 200 micrometers. In some embodiments, all of the particles have a size from about 10 micrometer to about 200 micrometers.

In some embodiments, at least 75% of the biodegradable polymer particles have a size from about 20 micrometer to about 180 micrometers. In some embodiments, at least 85% of the particles have a size from about 20 micrometers to about 180 micrometers. In some embodiments, at least 95% of the particles have a size from about 20 micrometer to about 180 micrometers. In some embodiments, all of the particles have a size from about 20 micrometer to about 180 micrometers.

In some embodiments, there is a pharmaceutical formulation comprising clonidine, wherein the clonidine is in a mixture of clonidine hydrochloride and clonidine base and the mixture comprises from about 0.1 wt. % to about 30 wt. % of the formulation and a biodegradable polymer comprises at least 70% of the formulation. In some embodiments, the polymer in this formulation is poly(L-lactide-co-caprolactone).

In some embodiments, the polymer is poly(L-lactide-co-caprolactone) or poly(D,L-lactide-co-caprolactone). In some embodiments, poly(L-lactide-co-caprolactone) has better handling characteristics (flexibility, reduced friability, ease of delivery, etc.) than poly(D,L-lactide-co-caprolactone) and allows the depot to be delivered for acute pain (e.g., post-operative pain). In various embodiments, poly(L-lactide-co-caprolactone) burst release is suitable for burst release and delivery of clonidine for post-operative pain. In some embodiments, the poly(D,L-lactide-co-caprolactone) does not have handling characteristics and a release profile suitable for treatment of pain (e.g., acute pain). For example, the burst release can be in some embodiments, about 5%, 10%, 15%, 20%, 25%, 30%, 35%, 45%, 50%, 55%, 60%, 65%, 70%, 75%, 80%, 85%, 90%, 95% to about 99% of the clonidine loaded in the depot over about 24 to 48 hours.

Mannitol, trehalose, dextran, mPEG and/or PEG may be used as a plasticizer for the polymer as well as the plasticizers and/or pore forming agents described above. In some embodiments, the polymer and/or plasticizer may also be coated on the depot to provide the desired release profile. In some embodiments, the coating thickness may be thin, for example, from about 5, 10, 15, 20, 25, 30, 35, 40, 45 or 50 microns to thicker coatings 60, 65, 70, 75, 80, 85, 90, 95, 100 microns to delay release of the drug from the depot (e.g., device). In some embodiments, the range of the coating on the depot ranges from about 5 microns to about 250 microns or 5 microns to about 200 microns to delay release from the device.

In various embodiments, the depot comprises poly(lactide-co-glycolide) (PLGA), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide), poly(L-lactide-co-ε-caprolactone), poly(D,L-lactide-co-ε-caprolactone) or a combination thereof and has an inherent viscosity of 0.2 to about 0.5 dL/g, or 0.4 to about 0.6 dL/g, or 0.6 to about 1.0 dL/gm and a MW of about 30,000 to about 125,000 Da.

In some embodiments, the depot comprises one or more polymers having a MW of from about 15,000 to about 150,000 Da, from about 25,000 to about 100,000 Da or from about 30,000 to about 50,000 Da or about 30,000 Da to about 60,000 Da.

In some embodiments, the depot comprises a polymer having an average molecular weight of the polymer can be from about 1,000 to about 10,000,000 Da; or about 1,000 to about 1,000,000 Da; or about 5,000 Da to about 500,000 Da; or about 10,000 Da to about 100,000 Da; or about 20,000 Da to 50,000 Da.

In some embodiments, when the polymer materials have different chemistries (e.g., high MW DLG 5050 and low MW DL), the high MW polymer may degrade faster than the low MW polymer.

As persons of ordinary skill in the art are aware, an implantable depot (e.g., device) compositions having a blend of polymers with different end groups are used the resulting formulation will have a lower burst index and a regulated duration of delivery. For example, one may use polymers with acid (e.g., carboxylic acid) and ester end groups (e.g., methyl or ethyl ester end groups).

Additionally, by varying the comonomer ratio of the various monomers that form a polymer (e.g., the L/G (lactic acid/glycolic acid) or G/CL (glycolic acid/polycaprolactone) ratio for a given polymer) there will be a resulting depot (e.g., device) composition having a regulated burst index and duration of delivery. For example, a depot (e.g., device) composition having a polymer with a L/G ratio of 50:50 may have a short duration of delivery ranging from about two days to about one month; a depot (e.g., device) composition having a polymer with a L/G ratio of 65:35 may have a duration of delivery of about two months; a depot (e.g., device) composition having a polymer with a L/G ratio of 75:25 or L/CL ratio of 75:25 may have a duration of delivery of about three months to about four months; a depot (e.g., device) composition having a polymer ratio with a L/G ratio of 85:15 may have a duration of delivery of about five months; a depot (e.g., device) composition having a polymer with a L/CL ratio of 25:75 or PLA may have a duration of delivery greater than or equal to six months; a depot (e.g., device) composition having a terpolymer of CL/G/L with G greater than 50% and L greater than 10% may have a duration of delivery of about one month and a depot (e.g., device) composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery. Thus, among other things, depot (e.g., device) compositions having a blend of polymers having different molecular weights, end groups and comonomer ratios can be used to create a depot (e.g., device) formulation having a lower initial burst and a regulated duration of delivery.

The depot (e.g., device) may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. If the depot (e.g., device) is to be placed in the spinal area, in various embodiments, the depot (e.g., device) may comprise sterile preservative free material.

The depot (e.g., device) can be different sizes, shapes and configurations. There are several factors that can be taken into consideration in determining the size, shape and configuration of the depot. For example, both the size and shape may allow for ease in positioning the depot at the target tissue site that is selected as the implantation. In addition, the shape and size of the system should be selected so as to minimize or prevent the depot from moving after implantation. In various embodiments, the depot can be shaped like a rod or a flat surface such as a film or sheet (e.g., ribbon-like) or the like. Flexibility may be a consideration so as to facilitate placement of the device.

In various embodiments, the depot can be different sizes, for example, the device may be a length of from about 0.5 mm to 50 mm and have a diameter of from about 0.01 to about 4 mm. In various embodiments, as the diameter decreases, the surface area that comes in contact with the bodily fluid of the depot (e.g., device) increases and therefore release of the drug from the depot (e.g., device) increases. In various embodiments, the device may have a layer thickness of from about 0.005 to 1.0 mm, such as, for example, from 0.05 to 0.75 mm. In various embodiments, the length of the device is determined based on the length needed to treat the target tissue site.

Radiographic markers can be included on the device to permit the user to position the depot (e.g., device) accurately into the target site of the patient. These radiographic markers will also permit the user to track movement and degradation of the depot (e.g., device) at the site over time. In this embodiment, the user may accurately position the depot (e.g., device) in the site using any of the numerous diagnostic imaging procedures. Such diagnostic imaging procedures include, for example, X-ray imaging or fluoroscopy. Examples of such radiographic markers include, but are not limited to, barium, phosphate, bismuth, iodine, tantalum, tungsten, and/or metal beads or particles. In various embodiments, the radiographic marker could be a spherical shape or a ring around the depot (e.g., device).

In some embodiments, a drug depot is provided that controls delivery of therapeutic agents to local, target tissues and secures itself to a target tissue site. In some embodiments, the drug depot is a flexible, drug loaded device that attaches to a target tissue site, such as, for example, muscle and/or fascia. In various embodiments, the device attaches to the target tissue site via adhesives that are applied to the entire device or at the ends of the device. In some embodiments, the drug depot is flexible, biodegradable devices or strands that are drug loaded and/or drug coated to provide sustained release of a therapeutic to a local tissue site. In some embodiments, drug release is in days to months. In some embodiments, the drug depot comprises polymers, such as, for example, 5:95 poly(D,L-lactide-co-caprolactone), 10:90 poly(D,L-lactide-co-caprolactone), 15:85 poly(D,L-lactide-co-caprolactone), 20:80 poly(D,L-lactide-co-caprolactone), 25:75 poly(D,L-lactide-co-caprolactone), 30:70 poly(D,L-lactide-co-caprolactone), 35:65 poly(D,L-lactide-co-caprolactone), 40:60 poly(D,L-lactide-co-caprolactone), 45:55 poly(D,L-lactide-co-caprolactone), 50:50 poly(D,L-lactide-co-caprolactone), 55:45 poly(D,L-lactide-co-caprolactone), 60:40 poly(D,L-lactide-co-caprolactone), 65:35 poly(D,L-lactide-co-caprolactone), 70:30 poly(D,L-lactide-co-caprolactone), 75:25 poly(D,L-lactide-co-caprolactone), 80:20 poly(D,L-lactide-co-caprolactone), 85:15 poly(D,L-lactide-co-caprolactone), 90:10 poly(D,L-lactide-co-caprolactone) or 95:5 poly(D,L-lactide-co-caprolactone).

Degradation times for the polymers could be days to 30 days. In some embodiments, the degradation time is about 2 weeks to about 30 days. In some embodiments, the degradation time is about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days or about 30 days.

In some embodiments, drugs are used such as, for example, an analgesic, anti-inflammatory and/or steroids, which are coated on the device or uniformly distributed throughout the device. In various embodiments, the device is memory shape device and can comprise shape memory polymers including various polyethers, polyacrylates, polyamides, polysiloxanes, polyurethanes, polyether amides, polyurethane/ureas, polyether esters, or urethanelbutadiene copolymers or a combination thereof.

The device provides a scaffold to release clonidine in vivo in three dimensions. In some embodiments, one or more devices may be stacked on one another.

In some embodiments, the device comprises a plurality of pores. In some embodiments, at least 10% of the pores are between about 10 micrometers and about 500 micrometers at their widest points. In some embodiments, at least 20% of the pores are between about 50 micrometers and about 150 micrometers at their widest points. In some embodiments, at least 30% of the pores are between about 30 micrometers and about 70 micrometers at their widest points. In some embodiments, at least 50% of the pores are between about 10 micrometers and about 500 micrometers at their widest points. In some embodiments, at least 90% of the pores are between about 50 micrometers and about 150 micrometers at their widest points. In some embodiments, at least 95% of the pores are between about 100 micrometers and about 250 micrometers at their widest points. In some embodiments, 100% of the pores are between about 10 micrometers and about 300 micrometers at their widest points.

In some embodiments, the device has a porosity of at least about 30%, at least about 50%, at least about 60%, at least about 70%, at least about 90%. The pore enhances release of the clonidine and supports pain relief to a patient after a surgical procedure.

In some embodiments, the initial burst surfaces can be disposed on the edges of the device so that upon contact with the target tissue site, the edges will begin to release the clonidine. In some embodiments, the body of the depot can comprise dense, entangled polymers and have the clonidine to provide slower release of the clonidine.

Alternatively, the clonidine can be disposed homogenously throughout the depot to provide continuous extended release of the clonidine. In some embodiments, the clonidine can be layered in the depot with some portions having different concentrations to provide burst release and then slower release of the clonidine in areas that have dense crosslinked polymers, such as for example, in the core of the device.

In some embodiments, the drug depot shown may have a burst release surface that releases about 5%, 10%, 15%, 20%, 25%, 30%, 35%, 45%, 50%, 55%, 60%, 65%, 70%, 75%, 80%, 85%, 90%, 95% to about 99% of the clonidine over about 24 to 48 hours.

In various embodiments, 1 to about 10 drug depots (e.g., pellets, strips, fibers, etc.) having an initial burst dose of about 5 to about 15% may be provided to a surgical site. In some embodiments, 1 drug depot may comprise an initial burst of about 5 to about 15% within 4 hours and about 10% to about 25% cumulative release in 24 hours to provide pain relief.

In some embodiments, the device may comprise natural and/or synthetic material. For example, the device may comprise poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters (POE), polyaspirins, polyphosphagenes, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyetheretherketone, polymethylmethacrylate, silicone, hyaluronic acid, chitosan, or combinations thereof.

In some embodiments, the device has a thickness of from 0.25 mm to 5 mm, or from about 0.4 mm to about 2 mm, or 0.4 mm to about 1 mm. In some embodiments, the device has a length of about 1 mm to about 300 mm or about 5 mm to 200 mm or about 5 mm to about 150 mm.

In some embodiments, the diameter of the device can range from 0.1 mm to 10 mm. In some embodiments, the diameter of the device can range from 0.1 mm to 5 mm, 0.1 mm to 3 mm or 0.1 mm to 1 mm.

In some embodiments, a variety of bioabsorbable polymers can be used to make the device. Examples of suitable biocompatible, bioabsorbable polymers include aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as elastin, bioabsorbable starches, etc.) or blends thereof. Polyesters include, but are not limited to, homopolymers and copolymers of lactide (which includes lactic acid, D-,L- and meso lactide), glycolide (including glycolic acid), caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one 2,5-diketomorpholine, pivalolactone, alpha-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one or polymer blends thereof.

In some embodiments, the depots may be of different sizes depending on the procedure being performed and the implant site. Depots (e.g., fiber, strips, pellet, etc.) can range in size from about 15 to about 35 mm.

In some embodiments, the depot may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

In some embodiments, a therapeutic agent (including one or more clonidine compounds) may be disposed on or in the device by hand by soaking, electrospraying, ionization spraying or impregnating, vibratory dispersion (including sonication), nozzle spraying, compressed-air-assisted spraying, brushing and/or pouring.

In some embodiments, the depot may comprise sterile and/or preservative free material. The depot can be implanted by hand or machine in procedures such as for example, laparoscopic, arthroscopic, neuroendoscopic, endoscopic, rectoscopic procedures or the like. In some embodiments, the depot can be made from a sponge material that can be spray coated or embedded with the clonidine, and as the sponge material degrades, the clonidine is released.

In some embodiments, the depot is in a cylindrical form, however, alternate shapes and configurations may be contemplated. In further exemplary embodiments, the depot may be a narrow tube for delivery through a catheter. For example, the depot may be delivered percutaneously using a catheter through which it is inserted. Thus, the depot may have dimensions suitable for receipt in the catheter. Optionally, the depot may be stiffened to facilitate insertion into the catheter. Such stiffening may be achieved through choice of material for the depot, by treating the material of the device, or other. In some embodiments, the depot may be coated with a material to facilitate sliding engagement with the catheter.

In certain embodiments, the length of the depot will range from about 15 mm to about 35 mm, the width will range from about 5 mm to about 15 mm, and the thickness will range from about 0.1 mm to about 0.3 mm. In some embodiments, the length may range from about 2.5 to about 15 mm, the width may range from about 1 to about 5 mm, and the thickness may range from about 5 mm to about 15 mm.

In some embodiments, the depot has a modulus of elasticity in the range of about 1×10² to about 6×10⁵ dynes/cm², or 2×10⁴ to about 5×10⁵ dynes/cm², or 5×10⁴ dynes/cm² to about 5×10⁵ dynes/cm².

In some embodiments, the semi-solid or solid depot 10 may comprise a polymer having a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.55 to about 0.6 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, or about 1.8 to about 2.1 dL/g.

In some embodiments, the depot may comprise a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethyl-methacrylate), poly-(methoxyethylmethacrylate), poly(methoxyethoxyethylmethacrylate), polymethyl-methacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In some embodiments, the depot may comprise gelatin, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. In some embodiments, the depot may comprise polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof.

In various embodiments, rather than directly admixing the therapeutic agent into the depot, microspheres may be dispersed within the device, the microspheres being loaded with clonidine. In one embodiment, the microspheres provide for a sustained release of the clonidine.

Microspheres, much like a fluid, may disperse relatively quickly, depending upon the surrounding tissue type, and hence disperse the clonidine. In some situations, this may be desirable; in others, it may be more desirable to keep the clonidine tightly constrained to a well-defined target site. The present invention also contemplates the use of adherent gel or adhesive to so constrain the device close to the target tissue site. In this embodiment, an adherent gel or adhesive is used to anchor the device to the target tissue site. The adherent gel or adhesive can, like the depot, also have the therapeutic agent disposed within it. In this way, the depot and the adhesive release the therapeutic agent (e.g., clonidine, statin, etc.) at or near the target tissue site.

### Device Delivery

It will be appreciated by those with skill in the art that the depot (e.g., device) can be administered to the target site using a “cannula” or “needle” that can be a part of a drug delivery device e.g., a syringe, a gun drug delivery device, or any medical device suitable for the application of a drug to a targeted organ or anatomic region. The cannula or needle of the device is designed to cause minimal physical and psychological trauma to the patient.

In some embodiments, the depot can be sutured to a target tissue site using a suturing needle. The dimensions of the needle, among other things, will depend on the site for implantation. For example, the width of the muscle planes in different surgical procedures can vary from 1-40 cm. Thus, the needle, in various embodiments, can be designed for these specific areas.

In various embodiments, like the depot, the cannula or needle includes dose radiographic markers that indicate location at or near the site beneath the skin, so that the user may accurately position the depot (e.g., device) at or near the site using any of the numerous diagnostic imaging procedures. Such diagnostic imaging procedures include, for example, X-ray imaging or fluoroscopy. Examples of such radiographic markers include, but are not limited to, barium, bismuth, tantalum, tungsten, iodine, and/or metal beads or particles.

In various embodiments, the needle or cannula may include a transparent or translucent portion that can be visualizable by ultrasound, fluoroscopy, X-ray, or other imaging techniques. In such embodiments, the transparent or translucent portion may include a radiopaque material or ultrasound responsive topography that increases the contrast of the needle or cannula relative to the absence of the material or topography.

The depot may be sterilizable. In various embodiments, one or more components of the device are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the device is included in a gel.

Other methods may also be used to sterilize the depot (e.g., device) and/or one or more components of the device, including, but not limited to, gas sterilization, such as, for example, with ethylene oxide or steam sterilization.

In various embodiments, a kit is provided that may include additional parts along with the depot combined together to be used to implant the depot. The kit may include the depot in a first compartment. The second compartment may include a canister holding the depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. A fifth compartment may include an agent for radiographic imaging. Each tool may be separately packaged in a plastic pouch that is radiation sterilized. A cover of the kit may include illustrations of the implanting procedure and a clear plastic cover may be placed over the compartments to maintain sterility. In some embodiments, a kit is provided with instruction to use an injectable drug from another kit.

In various embodiments, a method for delivering a therapeutic agent into a site of a patient is provided, the method comprising inserting a needle at or near a target tissue site and suturing the depot at the target site beneath the skin of the patient. In this way unwanted migration of the depot away from the target site is reduced or eliminated.

In some embodiments, the depot can be delivered to any site beneath the skin, including, but not limited to, at least one muscle, ligament, tendon, cartilage, spinal disc, spinal foraminal space, near the spinal nerve root, connective tissue, fascia, subcutaneous space, or spinal canal.

In some embodiments, it is preferable to co-administer clonidine with an antagonist to counteract undesirable effects, for example the blood pressure decrease that can be caused by clonidine. Exemplary antagonists include but are not limited to phentolamine, yohimbine, tolazoline and piperoxane. Additionally, compounds such as 5-fluorodeoxyuridine (FUDR) and 3,4 dehydroprolene may also be included. These compounds may prevent or reduce glial and fibroblastic scar formation associated with some types of surgeries.

Another embodiment is directed to a method for treating a mammal suffering from pain, said method comprising administering a therapeutically effective amount of clonidine at a target site beneath the skin. The clonidine (or pharmaceutically acceptable salt) may for example be administered locally to the target tissue site disposed within or on a device.

In some embodiments, the clonidine is encapsulated in a plurality of matrices comprising microparticles, microspheres, microcapsules, and/or microdevices and then put into a device.

In some embodiments there is a method for making an implantable device. The method may comprise combining a biocompatible polymer and a therapeutically effective amount of clonidine or a pharmaceutically acceptable salt thereof and forming the implantable device from the combination.

### Method of Making the Device

In various embodiments, the depot comprising the clonidine can be made by combining a biodegradable polymer and a therapeutically effective amount of clonidine or pharmaceutically acceptable salt thereof and forming the implantable device from the combination.

Various techniques are available for forming at least a portion of a depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot (e.g., device) to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: clonidine and other therapeutic agent(s) and other optional additives such as radiographic agent(s), etc. in dissolved or dispersed form. This results in a polymeric device region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot (e.g., device) or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: clonidine, optional additional therapeutic agent(s), radiographic agent(s), and so forth. The resulting mixture is then shaped into an implantable device. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, clonidine may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the clonidine containing device. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the device.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot (e.g., device) is formed comprising PLGA or PLA polymer, a radio-opacifying agent (e.g., bismuth subcarbonate), and a therapeutic agent prone to degradation by heat and/or mechanical shear (e.g., clonidine), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of clonidine because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot (e.g., device) is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing device (the pre-existing device can be formed using a variety of techniques including solution and thermoplastic processing techniques, and it can comprise a variety of additives including a radio-opacifying agent and/or viscosity enhancing agent), whereupon the therapeutic agent is imbibed on or in the device. As above, the resulting solid material can then be granulated for further processing, if desired.

Typically, an extrusion process may be used to form the device comprising a biocompatible polymer(s), therapeutic agent(s) and radio-opacifying agent(s). Co-extrusion may also be employed, which is a shaping process that can be used to produce a device comprising the same or different layers or regions (for example, a structure comprising one or more polymeric device layers or regions that have permeability to fluids to allow immediate and/or sustained drug release). Multi-region depots (e.g., devices) can also be formed by other processing and shaping techniques such as co-injection or sequential injection molding technology.

In various embodiments, the depot (e.g., device) that may emerge from the thermoplastic processing (e.g., pellet) is cooled. Examples of cooling processes include air cooling and/or immersion in a cooling bath. In some embodiments, a water bath is used to cool the extruded depot (e.g., device). However, where a water-soluble therapeutic agent such as clonidine is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot (e.g., device) surface, thus controlling or minimizing a high drug dose “initial burst” or “bolus dose” upon implantation or insertion if this is release profile is not desired.

In various embodiments, the device can be prepared by mixing or spraying the drug with the polymer and then molding the depot (e.g., device) to the desired shape. In various embodiments, clonidine is used and mixed or sprayed with the PLGA or PEG550 polymer, and the resulting depot (e.g., device) may be formed by extrusion and dried.

In various embodiments, there is a pharmaceutical formulation comprising: clonidine, wherein the clonidine comprises from about 0.1 wt. % to about 40 wt. % of the formulation, and at least one biodegradable polymer. In some embodiments, the clonidine comprises from about 3 wt. % to about 20 wt. %, about 3 wt. % to about 18 wt. %, about 5 wt. % to about 15 wt. %, 0.1 wt % to about 10 wt %, about 0.1 wt % to about 3 wt %, or about 7.5 wt. % to about 12.5 wt. % of the formulation. By way of example, when using a 5%-15% clonidine composition, the mole ratio of clonidine to polymer would be from approximately 16-53 when using an approximately 80,000 Da polymer that has a 267 grams/mole ratio. By way of another example, when using a 5%-15% clonidine base in the composition, the mole ratio of clonidine base to polymer would be from approximately 18-61 with a mole mass of 230 g/mol.

In some embodiments, the clonidine can be in the formulation in an amount of about 1%, 5%, 10%, 15%, 20%, 25%, 30%, 35%, or 40% by weight based on the total weight of the formulation.

In some embodiments, the device comprises at least one biodegradable material in a wt % of about 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 76%, 75%, 74%, 73%, 72%, 71%, 70%, 65%, 60%, 55%, 50%, 45%, 35%, 25%, 20%, 15%, 10%, or 5% based on the total weight of the depot (e.g., device) and the remainder is active and/or inactive pharmaceutical ingredients.

In some embodiments, at least 75% of the particles have a size from about 0.5 micrometer to about 100 micrometers. In some embodiments, at least 85% of the particles have a size from about 0.5 micrometers to about 100 micrometers. In some embodiments, at least 95% of the particles have a size from about 0.5 micrometer to about 100 micrometers. In some embodiments, all of the particles have a size from about 0.5 micrometer to about 100 micrometers. In some embodiments, at least 80% of the particles have a size from 2 microns to about 50 microns on a volume basis.

In some embodiments, the device comprises about 95 wt % poly(D,L-lactide) and 5 wt % clonidine HCl where the polymer has an ester end group and 50,000-70,000 Da MW and an IV 0.45-0.55 dL/g and has a burst release of under 10% of the amount of drug in the depot (e.g., device) within 24 hours (e.g., 5-10 wt %) or 2-40 mcg in 24 hours. This formulation has 50% of total cumulative dose remaining for at least 60 days. About 80% of the particles in this depot (e.g., device) including the clonidine are from about 5 to about 150 microns or 5-100 microns. The depot (e.g., device) releases about 0.5 mcg/day up to about 5 mcg/day of clonidine in 24 hours and then continues release for 70 days.

In some embodiments, the device comprises about 92 wt % poly(D,L-lactide) and 8 wt % clonidine HCl where the polymer has an ester end group and the polymer comprises 50,000-70,000 Da MW and an IV of about 0.45-0.55 dL/g and has a burst release of under 10% of the amount of drug in the depot (e.g., device) within 24 hours (e.g., 5-10%) or 5-6 mcg in 24 hours and then 1 to 20 mcg/day with a constant release for about 50 days, and then about 0.1 mcg to about 10 mcg/day for 70 days. This formulation has 50% of total cumulative dose remaining for at least 30-42 days and less than 80% cumulative drug release by 70 days. About 80% of the particles in this depot (e.g., device) including the clonidine are from about 5 to about 150 microns or 5-100 microns.

In some embodiments, the device comprises about 85 wt % poly(D,L-lactide) and 15 wt % clonidine HCl where the polymer has an ester end group and the polymer comprises 50,000-70,000 Da MW and an IV of about 0.45-0.55 dL/g and has a burst release of under 10% of the amount of drug in the depot (e.g., device) within 24 hours (e.g., 5-10%) or 20-150 mcg in 24 hours and then 5 to 80 mcg/day with a constant release for about 30 days, and then about 0.1 mcg to about 5 mcg/day for 70 days. This formulation has about 80% of total cumulative dose released within 35 days and 20% over several months. About 80% of the particles in this depot (e.g., device) including the clonidine are from about 5 to about 150 microns or 5-100 microns.

In some embodiments, there is a pharmaceutical formulation comprising clonidine, wherein the clonidine is in a mixture of clonidine hydrochloride and clonidine base and the mixture comprises from about 0.1 wt. % to about 30 wt. % of the formulation and a polymer comprises at least 70% of the formulation. In some embodiments, the polymer in this formulation is polyorthoester.

In some embodiments, the formulation comprises a device that comprises a biodegradable polyorthoester. The mechanism of the degradation process of the polyorthoester can be hydrolytical or enzymatical in nature, or both. In various embodiments, the degradation can occur either at the surface of the device (heterogeneous or surface erosion) or uniformly throughout the drug delivery system depot (e.g., device) (homogeneous or bulk erosion). Polyorthoester can be obtained from A.P. Pharma, Inc. (Redwood City, Calif.) or through the reaction of a bis(ketene acetal) such as 3,9-diethylidene-2,4,8,10-tetraoxospiro[5,5]undecane (DETOSU) with suitable combinations of diol(s) and/or polyol(s) such as 1,4-trans-cyclohexanedimethanol and 1,6-hexanediol or by any other chemical reaction that produces a polymer comprising orthoester moieties.

In some embodiment there is a higher loading of clonidine, e.g., at least 5 wt. %, at least 10 wt. %, at least 15 wt. %, at least 20 wt. %, at least 30 wt. %, at least 40 wt. %, at least 50 wt. %, or at least 60 wt. %.

A strategy of triangulation may be effective when administering these pharmaceutical formulations. Thus, a plurality (at least two, at least three, at least four, at least five, at least six, at least seven, etc.) devices comprising the pharmaceutical formulations may be placed around the target tissue site (also known as the pain generator or pain generation site) such that the target tissue site falls within a region that is either between the formulations when there are two, or within an area whose perimeter is defined by a set of plurality of formulations.

In some embodiments, the formulations are slightly rigid with varying length, widths, diameters, etc. For example, certain formulations may have a diameter of 0.50 mm and a length of 4 mm. It should be noted that particle size may be altered by techniques such as mort and pestle, jet-drying or jet milling.

The dosage of clonidine may be from approximately 0.0005 to approximately 960 μg/day. Additional dosages of clonidine include from approximately 0.0005 to approximately 900 μg/day; approximately 0.0005 to approximately 500 μg/day; approximately 0.0005 to approximately 250 μg/day; approximately 0.0005 to approximately 100 μg/day; approximately 0.0005 to approximately 75 μg/day; approximately 0.001 to approximately 70 μg/day; approximately 0.001 to approximately 65 μg/day; approximately 0.001 to approximately 60 μg/day; approximately 0.001 to approximately 55 μg/day; approximately 0.001 to approximately 50 μg/day; approximately 0.001 to approximately 45 μg/day; approximately 0.001 to approximately 40 μg/day; approximately 0.001 to approximately 35 μg/day; approximately 0.0025 to approximately 30 μg/day; approximately 0.0025 to approximately 25 μg/day; approximately 0.0025 to approximately 20 μg/day; approximately 0.0025 to approximately 15 μg/day; approximately 0.0025 to approximately 10 μg/day; approximately 0.0025 to approximately 5 μg/day; and approximately 0.0025 to approximately 2.5 μg/day. In another embodiment, the dosage of clonidine is from approximately 0.005 to approximately 15 μg/day. In another embodiment, the dosage of clonidine is from approximately 0.005 to approximately 10 μg/day. In another embodiment, the dosage of clonidine is from approximately 0.005 to approximately 5 μg/day. In another embodiment, the dosage of clonidine is from approximately 0.005 to 2.5 μg/day. In some embodiments, the amount of clonidine is between 40 and 600 μg/day. In some embodiments, the amount of clonidine is between 200 and 400 μg/day.

In some embodiments, the therapeutically effective dosage amount (e.g., clonidine dose) and the release rate profile are sufficient to reduce post-operative pain for a period of at least one day, for example, 1-2 days, 1-3 days, 1-4 days, 1-5 days, 1-6 days; 1-7 days, 1-8 days, 1-9 days, 1-10 days, 1-11 days, 1-12 days, 1-13 days, 1-14 days, 2-3 days, 2-4 days, 2-5 days, 2-6 days; 2-7 days, 2-8 days, 2-9 days, 2-10 days, 2-11 days, 2-12 days, 2-13 days, 2-14 days, 3-4 days, 3-5 days, 3-6 days; 3-7 days, 3-8 days, 3-9 days, 3-10 days, 3-11 days, 3-12 days, 3-13 days or 3-30 days.

In some embodiments the clonidine in the device is designed for a bolus dose or burst dose within 1, 2, or 3 days after implantation to provide an immediate release of the clonidine for treatment of pain and/or inflammation.

In some embodiments, the clonidine device is administered parenterally, e.g., by injection. In some embodiments, the injection is intrathecal, which refers to an injection into the spinal canal (intrathecal space surrounding the spinal cord). An injection may also be into a muscle or other tissue. In other embodiments, the clonidine depot (e.g., device) is administered by placement into an open patient cavity during surgery.

In some embodiments, there is a drug depot (e.g., device) comprising clonidine or clonidine hydrochloride and a polymer, wherein the polymer is one more of various embodiments, the drug depot (e.g., device) comprises poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone or a combination thereof.

In some embodiments, the polymer device of present application enables one to provide efficacy of the active ingredient that is equivalent to subcutaneous injections that deliver more than 2.5 times as much drug.

In some embodiments, the device comprises a polymer having 60 mol. % poly L-lactide and 40 mol. % caprolactone, where the poly(L-lactide-co-caprolactone) has a MW of 30,000 to 60,000 Da and an IV of about 0.4-0.6 dL/g and has a burst release of under 35% of the amount of drug in the depot (e.g., device) within 24 hours (e.g., 5-15% within 4 hours). The device comprises clonidine in an amount of 3-5 wt. %. The device releases 400 mcg to about 1000 mcg for 7 days, which is about 40 mcg/day. This device contains 1-10 wt % mannitol as a plasticizer and/or pore forming agent. The clonidine has a particle size of 10 to about 20 microns or less. The degradation time in the body is not more than 30 days and the device releases all of the clonidine within 2 to 14 days.

In some embodiments, the device comprises clonidine hydrochloride in an amount from about 3 wt. % to about 5 wt. % of the implantable medical device, poly(L-lactide-co-caprolactone comprising about 85 wt. % to about 95 wt. % of the implantable device, and mannitol in an amount from about 2.5 wt. % to about 10 wt. % of the implantable drug depot, the implantable drug depot is configured to release the clonidine hydrochloride over a period of at least 2 to about 14 days. As you drop the drug load the drug released from the depot (e.g., device) is faster. The clonidine is released in an amount between 80 ug and 250 ug per day for a period of about 2 to 14 days, the clonidine has a particle size of between about 10 to about 20 microns and the biodegradable depot degrades in about 30 days.

### Examples

The examples below with respect to certain formulations comprising clonidine as the biologically active agent show certain particularly advantageous results.

The inherent viscosity (IV) designations for the polymers are mentioned in Table A below. In some embodiments, the polymers can have the following inherent viscosities.

TABLE A

| IV Target Designator | IV Range |
|---|---|
| 1 | 0.05-0.15 |
| 1.5 | 0.10-0.20 |
| 2 | 0.15-0.25 |
| 2.5 | 0.20-0.30 |
| 3 | 0.25-0.35 |
| 3.5 | 0.30-0.40 |
| 4 | 0.35-0.45 |
| 4.5 | 0.40-0.50 |
| 5 | 0.45-0.55 |
| 6 | 0.50-0.70 |
| 7 | 0.60-0.80 |
| 8 | 0.70-0.90 |
| 9 | 0.80-1.0 |
| 10 | 1.0-1.2 |

The final letter within the code of the polymer is the end group designator. For examples “E” refers to an ester end group, while “A” refers to an acid end group.

By way of example, 100 LCL 5A is a polymer that has an inherent viscosity of about 0.45-0.55 dL/g. It contains 100% poly(L-lactide-co-caprolactone), where the ratio of L-lactide to caprolactone is 60:40 and has an ester end group. It is available from Lakeshore Biomaterials, Birmingham, Ala.

### EXAMPLES

The examples below with respect to certain formulations comprising clonidine as the biologically active agent show certain particularly advantageous results.

One embodiment of the device formulation is listed below in Table B.

### Example 1

### 14 Day in Vitro Elution of Clonidine Strip Formulations

In vitro elution testing was performed on various clonidine implant formulations. The implants were weighed and placed into cryotubes with 2 mL of 1×PBS (pH 7.4). The cryotubes were incubated in a shaker bath maintained at 37° C.+/−2° C. at 120 oscillations per minute. The in vitro release fluid was removed and replaced with fresh fluid at pre-determined intervals. The collected fluid was analyzed for clonidine content using reverse isocratic HPLC with UV detection. The results are summarized in FIG. 2. Results showed that the high dose strip (200-250 mcg/day for 2-5 days) had the best release for post-operative pain. The mid dose (150-200 mcg/day for 2-5 days) had the best dose for relieving acute pain (e.g. post-operative pain). The low dose (80 mcg/day for 2-5 days) had the least relief for acute pain (e.g., post-operative pain).

### Example 2

### 14 Day Pharmacokinetic Study in Beagle dogs

Clonidine strips were surgically implanted in intermuscular pockets in beagle dogs to evaluate the in vivo clonidine elution and plasma clonidine concentrations. Four male and four female dogs were divided into two groups of two (per sex). Group 1 received 6 strips of formulation 640-51 (5% clonidine HCl, 5% mannitol, 60:40 LCL 5A polymer, micronized API) and Group 2 received 6 strips of formulation 640-73 (4% clonidine HCl, 7.5% mannitol, 60:40 LCL 5A polymer, milled 25 micron API).

Blood was drawn at 30 minutes and 1, 2, 4, 8, and 24 hours post-implant. Blood was then drawn daily through day 7, and again at days 10 and 14. The blood was processed to plasma and the clonidine concentration in the plasma was determined by high performance liquid chromatography and tandem mass spectroscopy (LC/MS/MS). The results are summarized in FIG. 3. Results showed that the 640-73 formulation (4% clonidine HCl, 7.5% mannitol, 60:40 LCL 5A polymer, milled 25 micron API) had the fastest release (increased burst release) but did not last as long as the other formulations. The 640-51 formulation (5% clonidine HCl, 5% mannitol, 60:40 LCL 5A polymer, micronized API) had a lower burst release and lasted longer in dogs according to plasma concentrations. It was surprising that the lower dosage of clonidine of a 4% drug load had a higher burst release. Mannitol speeds up drug release within the first 72 hours.

### Example 3

### 7 Day Analgesic Efficacy Study in the Porcine Flank Incisional Model

Various clonidine implant formulations were evaluated for analgesic efficacy over a period of 7-days post-surgery using a porcine flank-incision model. Efficacy was addressed by measuring mechanical allodynia in response to Von Frey filament stimuli near the incision area.

The flank-incision model involves creating a 6-cm skin and fascia incision that doesn't involve the muscle, as shown in FIG. 1. A sham surgical procedure group was used as the negative control. The positive control group received daily, peri-incisional bolus injections of clonidine solution (1 mL injected, 225 micrograms/mL). The clonidine implant groups used various formulations and numbers of strips (1 to 3 strips) to target low dose, middle dose, and high dose groups. The clonidine implants were placed end to end as a single layer in the incisional space on Day 0 prior to incision closure. Sustained release clonidine implant treatments targeted the following daily doses of clonidine:

Low Dose: ˜150-200 micrograms on Day 1, followed by ˜80 micrograms/day Days 2-5
Mid Dose: ˜300-450 micrograms on Day 1, followed by ˜150-200 micrograms/day Days 2-5
High Dose: ˜500-600 micrograms on Day 1, followed by ˜200-250 micrograms/day Days 2-5

TABLE B

| Group Size | Treatment |
|---|---|
| 8 | Surgery only |
| 8 | Low Dose |
| | (~80 ug/d) |
| 8 | Mid Dose |
| 8 | (~150-200 ug/d) |
| 8 | |
| 8 | High Dose |
| 8 | (~200-250 ug/d) |
| 7 | Daily Injection |
| | (225 ug/injection/day) |

Analgesic effect was assessed using the Von Frey methodology. Von Frey filaments were applied 0.5-1.0 cm away from the incision line at various time points post-implantation. The Von Frey force (g) data were normalized by logarithmic transformation before statistical data analyses were performed. The effect of each treatment was assessed by comparing its mean log-transformed Von Frey force data to that of the untreated group. The low dose group demonstrated only slight improvement in pain relief from the untreated group at Day 1, Day 4 and Day 5 (p<0.05). The mid and high dose groups demonstrated significant improvement in pain relief from the untreated group at all time points from Day 0 through Day 5 (p<0.01 or better).

### Example 4

### Exemplary Formulation

TABLE C

| | |
|---|---|
| 1) Clonidine HCl content: | 3-5 wt % (4% nominal) |
| 2) Mannitol content: | 2.5-10 wt % (7.5% nominal) |
| 3) Poly(L-lactide-co-ε- | 85-94.5 wt % |
| caprolactone): | |
| a. Mole ratio range: | 60:40 to 70:30 L-CL |
| b. Inherent viscosity: | 0.4-0.6 dL/g (nominal 0.5 dL/g) |
| 4) The MW of the product | 30,000 Da-60,000 Da |
| (not the polymer raw material): | (nominal ~45,000 Da) |

One exemplary formulation is listed above in Table C. Mannitol was used as a plasticizer and/or pore forming agent. Mannitol at an amount of about 1% to about 30% of the total weight of the depot micronizes the polymer and has a particle size of about 1 to about 250 microns, about 1 to about 100 microns or about 1 to about 10 microns.

The exemplary formulation provides mannitol at an amount of 2.5-10 wt % added to a drug depot (e.g., strip, pellet, fiber, etc.). Results show that mannitol at 2.5-10 wt % provides an optimal burst release for clonidine HCL at 3-5 wt % at an incision site, providing pain relief to a patient after the incision site is repaired.

Results show that poly(L-lactide-co-ε-caprolactone) has optimum handling characteristics that allow the depot to be delivered for post-operative pain. Burst release is suitable for burst release and delivery of clonidine for post-operative pain.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Having now generally described the invention, the same may be more readily understood through the following reference to the following examples, which are provided by way of illustration and are not intended to limit the present invention unless specified.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An implantable medical device for reducing or treating post-operative pain in a patient in need of such treatment, the implantable medical device comprising clonidine in an amount from about 1 wt. % to about 30 wt. % of the implantable medical device, and at least one biodegradable polymer and a pore forming agent in an amount from about 1 wt. % to about 30 wt. % of the implantable medical device, wherein the implantable medical device is configured to release the clonidine over a period of at least 48 hours.

2. An implantable medical device according to claim 1, wherein the implantable medical device is a drug depot and the biodegradable polymer comprises about 70 wt. % to about 98 wt. % of the implantable medical device.

3. An implantable medical device according to claim 1, wherein the clonidine is released in an amount between 80 ug and 250 ug per day for a period of about 2 to 14 days.

4. An implantable medical device according to claim 1, wherein the clonidine comprises a salt comprising magnesium, potassium, ammonium, hydriodic, hydrobromic, phosphoric, metaphosphoric, nitric, sulfuric acids, hydrochloric acid, tartaric, acetic, citric, malic, benzoic, glycollic, gluconic, gulonic, succinic, arylsulfonic or p-toluenesulfonic acids.

5. An implantable medical device according to claim 1, wherein the clonidine comprises clonidine hydrochloride in an amount of about 3 wt. % to about 5 wt. %.

6. An implantable medical device according to claim 1, wherein the implantable medical device comprises a plasticizer in an amount of about 1 wt. % to about 10 wt. % of the implantable medical device.

7. An implantable medical device according to claim 1, wherein the biodegradable polymer comprises poly(L-lactide-co-caprolactone), poly(lactic-co-glycolide) (PLGA) or poly(orthoester) (POE), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(D-lactide-co-caprolactone), poly(D,L-lactide), poly(D-lactide), poly(L-lactide), poly(esteramide) or a combination thereof.

8. An implantable medical device according to claim 1, wherein the biodegradable polymer comprises poly(L-lactide-co-caprolactone) in an amount of about 85 wt. % to about 95 wt. % of the implantable medical device.

9. An implantable medical device according to claim 8, wherein the poly(L-lactide-co-caprolactone) has a molar ratio of about 60:40 to about 70:30 of lactide to caprolactone.

10. An implantable medical device according to claim 8, wherein the poly(L-lactide-co-caprolactone) has an inherent viscosity of about 0.4 to about 0.6 dL/g.

11. An implantable medical device according to claim 8, wherein the poly(L-lactide-co-caprolactone) has a molecular weight of about 30,000 Da to about 60,000 Da.

12. An implantable medical device according to claim 1, wherein the pore forming agent comprises MgO, mPEG, propylene glycol, mannitol, trehalose, TBO-Ac, Span-65, Span-85, pluronic F127, sorbitol, isomalt, erithlitol, cyclodextrin, maltodextrin, pluronic F68, CaCl, dextran, dextran sulphate, dextran phosphate, hydroxypropylcellulose, ethylcellulose, PEG 1500, PEG 400, PEG3350 or combinations thereof.

13. An implantable medical device according to claim 1, wherein the pore forming agent comprises mannitol in an amount of about 2.5 wt. % to about 10 wt. %.

14. An implantable medical device according to claim 1, wherein the clonidine comprises clonidine hydrochloride in an amount of about 1 wt. % to about 5 wt. %., wherein the biodegradable polymer comprises poly(L-lactide-co-caprolactone) in an amount of about 85 wt. % to about 95 wt. % of the implantable medical device, and having a molar ratio of about 60:40 to about 70:30, and having an inherent viscosity of about 0.4 to about 0.6 dL/g, and a molecular weight of about 30,000 Da to about 60,000 Da, and the pore forming agent comprises mannitol in an amount of about 2.5 wt. % to about 10 wt. %.

15. An implantable medical device according to claim 1, wherein the clonidine has a particle size of between about 10 to about 20 microns.

16. An implantable medical device according to claim 1, wherein the biodegradable depot degrades in about 30 days.

17. An implantable medical device for treating post-operative pain in a patient in need of such treatment, the implantable medical device comprising clonidine hydrochloride in an amount from about 3 wt. % to about 5 wt. % of the implantable medical device, poly(L-lactide-co-caprolactone) comprising about 85 wt. % to about 95 wt. % of the implantable medical device, and mannitol in an amount from about 1 wt. % to about 10 wt. % of the implantable medical device, wherein the implantable medical device is configured to release the clonidine hydrochloride over a period of at least 2 to about 14 days.

18. An implantable medical device according to claim 17, wherein the implantable medical device is a drug depot having a burst release of the clonidine hydrochloride of from about 55% to about 85% over a period of about 3 to 10 days after the depot is implanted beneath skin.

19. A method for treating post-operative pain in a patient in need of such treatment, the method comprising: administering an implantable medical device beneath the skin of a patient, the implantable medical device comprising clonidine in an amount from about 1 wt. % to about 30 wt. % of the implantable medical device, and at least one biodegradable polymer and a pore forming agent in an amount from about 1 wt. % to about 30 wt. % of the implantable medical device, wherein the implantable medical device is configured to release the clonidine over a period of at least 48 hours.

20. A method for treating post-operative pain according to claim 19, wherein the biodegradable polymer comprises about 70 wt. % to about 98 wt. % of the implantable medical device and the clonidine is released in an amount between 80 ug and 250 ug per day for a period of about 2 to 14 days.
